(19) United States
(12) Patent Application Publication
(10) Pub. No.: US 2024/0292119 A1
(43) Pub. Date: Aug. 29, 2024

(54) IMAGING APPARATUS AND METHODS WITH DETECTOR HAVING STACKED WIRING LAYERS PROVIDING FAST READOUT

(71) Applicant: REAL TIME IMAGING TECHNOLOGIES, LLC, Charlotte, NC (US)

(72) Inventor: Daniel UZBELGER FELDMAN, Charlotte, NC (US)

(21) Appl. No.: 18/380,530

(22) Filed: Oct. 16, 2023

Related U.S. Application Data
(60) Provisional application No. 63/539,809, filed on Sep. 21, 2023.
(60) Provisional application No. 63/486,474, filed on Feb. 23, 2023.

(51) Int. Cl. H04N 25/30; H04N 25/74; H04N 25/766; H04N 25/773; H04N 25/78

(52) U.S. Cl. CPC H04N 25/30; H04N 25/74; H04N 25/766; H04N 25/773; H04N 25/78; G01T 1/247

(57) ABSTRACT

An imaging method includes the steps of: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. The detector includes a plurality of wiring layers having stacked substrates attached together. Furthermore, each substrate includes one or more processing circuits, by which the detector is configured for fast readout speed and dual native ISO.

### COPYRIGHT STATEMENT

Any new and original work of authorship in this document is subject to copyright protection under the copyright laws of the United States and other countries. Reproduction by anyone of this document as it appears in official governmental records is permitted, but otherwise all other copyright rights whatsoever are reserved.

### BACKGROUND OF THE INVENTION

The present invention in general relates to x-ray imaging apparatus and methods and, in particular, to such imaging apparatus and methods that have a fast readout speed, and low-noise multiple signal amplification. Preferred embodiments relate to such apparatus and methods used in the context of dental and medical x-ray imaging modalities.

The demand is still increasing for commercial, cinema and scientific cameras, as well as for smartphones, which have high speed, low-noise, and ultra-high-definition (UHD) for resolutions beyond 4K CMOS image sensors. The high speed is desirable for slow-motion applications and a reducing rolling shutter distortion for moving objects and subjects. The low-noise is desirable for capturing a clear image in low-light conditions.

New advances in digital cameras and smartphones now allow for faster readout speeds thanks to the introduction of the stacked back illuminated CMOS sensor, as shown in U.S. Pat. Nos. 5,773,379 and 8,773,562. Through this approach, digital cameras and smartphones can offer faster frames per second while minimizing the blur effect from moving objects and subjects in a still picture. To achieve this further, all components are positioned in a similar arrangement, but the design stacks the image signal processor, circuits and its ultra-fast dynamic random-access memory (DRAM) in additional layers into the same silicon instead of laterally or at the periphery. This allows for higher bandwidth, lower delays and, in the end, faster data transfer. In addition, low noise in low light conditions has been improved thanks to the incorporation of dual native ISO conversion gain to commercial digital cameras and smartphones, as shown in U.S. Pat. Nos. 7,075,049 and 8,648,287. “ISO” is a term that was originally used in film cameras and derived from the International Organization for Standardization (ISO), the German Standards Organization (DIN), and the American Standards Association (ASA), and represents the sensitivity of film and digital cameras to light (or ISO speed). ISO divided the scale of film to be sensitive to different levels of light. At the typical digital camera setup in professional photography and videography, the lens aperture function, the shutter speed, the exposure value, and the ISO settings play a pivotal role for image acquisition. The native ISO is the point where the sensor achieves its best signal-to-noise ratio (SNR) which is the point where the noise is the least and the camera can produce the best combination of color, contrast, dynamic range (DR), and other qualities. In the way commercial cameras use this dual native ISO approach, low-light images that otherwise would produce a higher noise in a higher ISO setting can extend their DR while reducing the signal noise that is produced. The camera can then read two native ISOs on the sensor instead of one. In these cameras, rather than one set of circuits adjusting the processing and transmitter to achieve a native ISO, there are two. Thus, while one native ISO may be 800, a second might be 3200.

In order to achieve an UHD image resolution in the commercial cameras and smartphones world, a wider dynamic range (WDR) performance without SNR drop at the signal switching point is desired for improving sensing accuracy over the wide range of illumination conditions and ISO settings. In addition, suppression of motion blur is important when capturing moving objects and subjects. Several WDR technologies have been reported so far, including multiple exposure, dual photodiode (PD), dual conversion gain (DCG), lateral overflow integration capacitors (LOFICs), two-stage LOFICs, binary pixels, and combinations of the foregoing. The multiple exposure approach captures a few images with different exposure periods. The approach with dual PDs acquires an image with multiple PDs with different size or light sensitivity in a single exposure. The DCG approach changes conversion gain (CG) by controlling a switch connected to a capacitor and floating diffusion (FD) in a pixel during the horizontal blanking period. The LOFIC approach accumulates overflow-electrons from PD and FD capacitors and reads out signals with different sensitivity in a single exposure. This approach allows the independent design of CG and full well capacity (FWC), resulting in about 100 dB single exposure DR. The two-stage LOFICs allows increasing the DR while reducing sensor's pixel size. In a similar approach, the binary pixels DR extension oversamples the incident light by using the ADC output at each sampling and resetting the pixel only if a threshold has been exceeded. Consequently, the maximum SNR on the high light side can be increased without decreasing sensitivity. In addition, single PD architecture is beneficial to maintain the optical performance when applying the image sensors to the various optical configurations.

Many imaging apparatus and methods are known. Current 2D and 3D dental x-ray imaging, dental fluoroscopy, digital tomosynthesis, photon-counting computed tomography, PET/SPECT scan devices, and general radiography, fluoroscopy, mammography, computed tomography and CBCT scan devices, all comprise front illuminated sensors. A traditional front-illuminated sensor is constructed in a fashion similar to the human eye, with a pixel gate and a digital matrix at the front and photosensitive areas at the back. This traditional orientation of the sensor places the active matrix of the digital image sensor—a matrix of individual picture elements—on its front surface and simplifies manufacturing. The matrix and its wiring, however, reflect some of the light, and thus the photosensitive areas can only receive the remainder of the incoming light; the reflection reduces the signal that is available to be captured resulting in a decrease in sensor's quantum efficiency (QE). Consequently, front illuminated systems require increased radiation doses and large pixel sizes in order to gather enough light to produce an image. Patent references disclosing dental and medical imaging apparatus and methods following these principles include U.S. Pat. Nos. 4,057,727; 5,434,418; 5,834,782; 6,972,411; 7,016,461; 7,136,452; 7,197,109; 7,319,736; 7,322,746; 7,323,692; 7,336,763; 7,426,258; 7,563,026; 7,596,205; 7,608,834; 7,615,754; 7,629,587; 7,653,263; 8,378,310; and 9,180,215.

Current intraoral and extraoral digital dental x-ray system comprise an x-ray source with a milliamperes (mA) setting ranging in between 2.5 and 15 mA, a kilovolt peak (kVp) ranging in between 60 and 120 kVp and exposure times ranging in between 0.016 and 1.65 seconds for intraoral, and up to 20 seconds for extraoral. It is believed that intraoral dental systems did not allow frames per second until this feature was introduced through Uzbelger Feldman U.S. Pat. Nos. 6,543,936 and 8,430,563, and then for low-dose radiography through, for example, Uzbelger Feldman U.S. Pat. Nos. 10,849,586, 10,898,070 and 11,559,268, each of which are incorporated herein by reference. The apparatuses and methods disclosed therein exhibited increased sensor QE and fill factor for a lower dose over conventional devices, while allowing a pixel size reduction for an improved image resolution.

Thanks to the above efforts, the milliamperes (mA) range has been taken into consideration in the attempts to reduce the radiation doses to which dental patients are exposed. The milliamperage is the units of the electrical current that is used to produce the radiation dose. The quantity, or number of x-rays emitted from the tube head, is controlled by the mA. With all other technical factors (e.g., kVp, time) held constant, patient radiation dose is directly proportional to the mA. A 50% in mA reduction results in a decrease in radiation dose by 50%. Radiation reduction in dentistry has been proposed through the introduction of a low-dose static x-rays and fluoroscopy technology by minimizing the mA settings and the use of image intensification, as described in the Uzbelger Feldman '936 patent and '563 patent.

In particular, these recent breakthroughs in dose reduction made possible the production of low mA settings for dental use as disclosed in the Uzbelger Feldman '936 patent by using small image intensifiers in between a detector's converter/plate and collector. Despite these efforts, detector's configuration using the image intensifier and collector was still too bulky to be used inside the mouth and not ergonomic for the dentist to be placed extraorally while performing treatments on patients. Another disadvantage of intensifiers is image distortion originating from the projection of the x-ray image onto the curved input phosphor, and a smaller component corresponding to the mapping from the input phosphor to the output phosphor and the digital image matrix. In order to overcome these obstacles, a current attempt of on-chip image intensification for low milliamperes image capturing is known from the Uzbelger Feldman '563 patent by amplifying the electrical signals within the detector's collecting area reducing a need for image intensifier coupling; however, in this approach no image collection description at the photosensitive areas without passing through the wiring layer of the sensor is disclosed or suggested. As a result, this system would require a large pixel size ranging from 100 to 200 um. In addition to the single wiring layer within the collector, there is only one circuit dedicated to electrical signal amplification.

A recent attempt described in the Uzbelger Feldman '070 patent is believed to lead to increased low light collection efficiency in dental and medical radiology. In this regard, a microlens array is incorporated in the detector for collecting and focusing light, which would have otherwise fallen onto the non-sensitive areas of the collector. The use of a microlens array in imaging detectors in between the converter/plate and the collector is believed to help increase image QE without compromising sensor's size and image resolution. It is also seen to be advantageous in that there is no necessity to couple or attach additional bulky components, as a microlens array is very thin and fits well within a detector. The use of a microlens array also contributes to system's pixel size reduction for an improved spatial resolution image.

Additional efforts to increase the light collection are disclosed in the Uzbelger Feldman '586 patent through a novel back illuminated pixel architecture. Thinned back illuminated CMOS sensor principles as disclosed in U.S. Pat. No. 4,266,334 are believed to have been invented around 1981. It is credited that the back illuminated CMOS commercial adoption started when OmniVision developed their first sensors using the technique in 2007. The first widely used back illuminated sensor was the OmniVision OV8810, which was announced in 2008, and comprised 8 megapixels and a 1.4 μm pixel size. The OV8810 was used in the HTC Droid Incredible and the HTC EVO 4G. Back illuminated sensors currently are used in digital cameras and smartphones for capturing images in low light conditions, such as disclosed in U.S. Pat. No. 7,521,335. In addition, the use of back illuminated CMOS sensors has been suggested for x-ray astronomy imaging purposes in U.S. Pat. No. 8,575,559; for x-ray diffraction detection and radiation monitoring in U.S. Pat. No. 8,421,007; for medical computed tomography in U.S. Pat. Nos. 10,761,219; 9,588,239; 8,288,733; 8,121,248; 7,869,559; 7,620,143; 7,455,454; and 6,426,991; and, for x-ray spectrometry purposes in U.S. Pat. No. 4,245,158.

Newer developments describe a stacked back illuminated single-photon avalanche diode (SPAD) array sensor developed by Apple and disclosed in U.S. patent application publication 2018/0090536. Its main advantage over CMOS is low-light detection near-infrared (NIR) region. It is believed that the world's first commercial NIR stacked back-illuminated SPAD was implemented in the iPad 11 Pro in 2019. SPAD technologies have also been proposed for x-ray imaging detectors in U.S. Pat. No. 11,696,865 and in the Korean patent publication KR20230100090A of 2023 in which a stacked back illuminated approach is not described.

Despite of these advancements and a dose reduction approach, data readout has space constraints laterally or at the sensor periphery while signals' amplification can only be achieved through a single set of circuits within the system. This happens because a standard back illuminated CMOS sensor has one layer in which the pixels and associated wiring circuitries are grouped together, and as a result, the primary task of collecting the light and processing is done within that layer, as disclosed in the Uzbelger Feldman '586 patent and '268 patent.

In view of the foregoing, additional improvements are believed to be desirable over the aforementioned imaging apparatus and systems, including all dental and medical x-ray imaging modalities such as intraoral and extraoral 2D and 3D dental imaging, digital tomosynthesis, photon-counting computed tomography, photon-counting radiography, PET/SPECT scans, general radiography, fluoroscopy, dental fluoroscopy, mammography, computed tomography and CBCT. In particular, it is believed that needs exist for a detector with higher megapixel counts that has improved readout speed, a detector that has lower-noise multiple signal amplification, and especially, a detector that has both improved readout speed and lower-noise multiple signal amplification. One or more aspects and features of the invention address one or more such needs.

### SUMMARY OF THE INVENTION

The present invention includes many aspects and features. Moreover, while many aspects and features relate to, and are described in, the context of dentistry and medicine, the present invention is not limited to use only in such context, as will become apparent from the following summaries and detailed descriptions of aspects, features, and one or more embodiments of the present invention. Other fields that can benefit from the present invention include veterinary medicine, astronomy, industrial x-ray inspection, non-destructive testing, and airport security.

In an aspect of the invention, an imaging method comprises the steps of: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. In this aspect, the detector comprises a plurality of wiring layers comprising stacked substrates attached together, each substrate comprising one or more processing circuits by which the detector is configured for fast readout speed and dual native ISO.

In a feature, the stacked substrates of the wiring layers are attached together by microbumps, direct bonding followed by Via-last through silicon via (Via-last TSV), hybrid bonding (HB) technologies or a combination thereof.

In a feature, each of the stacked substrates of the wiring layers comprise a single wafer or a plurality of wafers. The wafers may be stitched, butted, or both.

In a feature, the detector comprises two or more wiring layers each comprising a said substrate.

In a feature, the plurality of wiring layers is oriented behind one or more photosensitive areas in the direction of travel of the light within the detector.

In a feature, the stacked substrates of the wiring layers comprise one or more DRAM; WDR logic; readout circuitry; one or more analog-to-digital converters with single or hybrid column counter and a scalable low voltage signaling interface with an embedded clock (SLVS-EC) or a SLVS with a double data rate source-synchronous clock (DDR-SSC); a column parallel correlated multiple sampling (CMS) effects readout circuits with one or more output streams for controlling the switching of sub streams at each frame; one or more digital to analog converter (DAC); and, line buffers. For the stacked substrate of the SPAD, the wiring layers and pixel electronics could be a simple SPAD, a SPAD with a bit counter, a SPAD with a bit counter and a time-to-digital converter (TDC) or a combination thereof.

In a feature, the stacked substrates of the wiring layers comprise a plurality of analog-to-digital converters, and wherein the data is transmitted using the plurality of analog-to-digital converters for single or parallel multiple sampling readout and one or more output streams for controlling the switching of sub streams at each frame. The computing device to which the data is transmitted preferably is a desktop or laptop computer, a wireless mobile computing device, a tablet, or smartphone, VR glasses, a headset, or a hologram projector. The method also preferably comprises the steps of receiving the fast readout speed and dual native gain transmitted data and processing the data and displaying the digital images to a user on the computing device. The digital images displayed to a user may be 2D and 3D still images or real time video.

In a feature, the method further comprises aiming the light to the photosensitive areas of the collector within the detector. The aiming of the light preferably is performed using a microlens array, an anti-glare filter, a light intensity boost film, a light control film or a color filter, a radiation hardened or non-radiation hardened fiber optic plate or nano optic plate, or combination thereof.

In a feature, the stacked substrates of the wiring layers comprise a radiation resistant chip.

In a feature, the collector has a pixel size from 0.001 microns to 500 microns.

In a feature, transforming the beam into light is performed by one or more organic x-ray converters, an organic photoconductive film (OPF), an organic photodetector (OPD), inorganic x-ray converters, or combination thereof, including Perovskite, halide Perovskite (inorganic, hybrid, organic-inorganic, 2/3D mixed dimensional and double Perovskite), lead halide Perovskite and single-crystalline Perovskite.

In a feature, the x-ray converter comprises a scintillator, a nanodots-based converter or a combination thereof.

In a feature, the x-ray converter comprises a nanodots-based converter that is made from one of the groups of inorganic quantum dots, carbon-based quantum dots, perovskites quantum dots or a combination thereof.

In a feature, the x-ray converter comprises a solid, liquid, gas, or combination thereof, a said x-ray converter is coupled to the collector; the x-ray converter is coupled to a plate and the plate is coupled to the collector; and combinations thereof.

In a feature, the collector acts as an x-ray converter, such as in a direct radiography approach, photon counting, or a combination thereof.

In a feature, the beam caused to be emitted comprises a low-dose gamma ray or X-ray beam. The emitter preferably comprises one or more x-ray tubes, one or more gamma ray sources, or a combination thereof. With respect to the x-ray tubes, each x-ray tube preferably comprises a filament-based tube or a cold cathode-based tube such as a carbon nanotube having one or more focal spot sizes ranging from 0.001 microns to 3 mm. The nano focus and microfocus focal spot sizes will allow to achieve high resolution imaging at the cellular level for detecting early-stage disease.

In a feature, the detector comprises a lightproof housing within which components of the detector are enclosed. The lightproof housing preferably comprises a radiation shielded back side that minimizes backscattered radiation.

In a feature, the detector comprises an intraoral or internal detector.

In a feature, the detector comprises an extraoral or external detector.

In a feature, the detector comprises an X-ray line detector with time-delay-integration (TDI).

In a feature, the detector comprises a scalable tile detector.

In a feature, the detector comprises a flat panel detector.

In a feature, a housing of the detector comprises a coating layer that filters or reflects the light within the detector. The coating layer preferably is located on top of a converter.

In a feature, the method is used in dental digital radiography; low-dose dental digital radiography; dental fluoroscopy; dental panoramic scanning; dental cephalometric scanning; dental cone beam computed tomography (CBCT); linear tomography; digital tomosynthesis; dental x-ray stereoscopic spectroscopy; photon-counting computed tomography; photon-counting radiography, positron emission tomography (PET); single-photon emission computed tomography (SPECT); and general radiography, fluoroscopy, mammography and computed tomography.

In another aspect, an imaging method comprises the steps of: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. The collector comprises a stacked, back-illuminated sensor having two or more wiring layers by which the detector is configured for fast readout speed and dual native ISO.

In an aspect, an imaging method comprises: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. The detector comprises a plurality of wiring layers comprising stacked substrates attached together, each substrate comprising one or more processing circuits including amplifiers and one or more analog-to-digital converters, the amplifiers amplifying the electrical signals within the detector before being processed by the one or more analog-to-digital converters for single or parallel multiple sampling readout and one or more output streams for controlling the switching of sub streams at each frame via multiple exposure, dual PD, two-stage LOFICs, binary pixels, or combinations thereof, to allow a dual native gain while minimizing SNR and maintaining a WDR.

In an aspect, a method of making a stacked, back-illuminated sensor comprises: (a) flipping a silicon wafer of a conventional detector so as to orientate two or more wiring layers behind the one or more photosensitive areas of the wafer with respect to a direction of incident light to be received, and (b) thinning a side receiving the incident light so that the incident light strikes the one or more photosensitive areas of the wafer without passing through the two or more wiring layers of the wafer.

In an aspect, a low-dose radiation imaging apparatus comprises: (a) one or more emitters each configured to emit a low-dose gamma or x-ray beam through a patient examination area; and (b) one or more detectors each configured to receive a said beam. Each detector comprises a housing containing: (i) a collector that converts the light into electrical signals representative of digital images corresponding to the patient examination area, wherein the light that is collected passes to one or more photosensitive areas of the collector without passing through any wiring layer of the collector, and (ii) a transmitter that transmits from the detector the data representative of digital images for display of the digital images to a user on a computing device. The two or more wiring layers comprise the transmitter and further comprise one or more DRAM, WDR logic, readout circuitry, one or more analog-to-digital converters with single or hybrid column counter with a scalable low voltage signaling interface with an embedded clock (SLVS-EC) or a SLVS with a double data rate source-synchronous clock (DDR-SSC), a column parallel correlated multiple sampling (CMS) effects readout circuit with one or more output streams for controlling switching of sub streams at each of a plurality of frames, digital to analog converter (DAC), and line buffers, For the stacked substrate of the SPAD, the wiring layers and pixel electronics could be a simple SPAD, a SPAD with a bit counter, a SPAD with a bit counter and a time-to-digital converter (TDC) or a combination thereof.

As a result, a large number of frames per second and greater image acquisition is achieved with a reduction in radiation dosage, a blur effect from moving objects and subjects during a radiographic examination or fluoroscopic procedures is minimized, seamlessly switching from video to still picture and vice versa, and slow-motion displaying are provided.

In a feature, the one or more emitters, the one or more detectors, or both, are configured to rotate around the examination area.

In a feature, one or more emitters, one or more detectors, or combination thereof are mounted to a wall or ceiling by mechanical arms, c-arms, u-arms or o-arm supports.

In a feature, one or more emitters, one or more detectors, or combination thereof are attached to a fixed gantry.

In a feature, one or more emitters, detectors, or both are handheld and portable.

In a feature, two or more emitters are configured to direct beams to the same detector.

In a feature, the beam caused to be emitted comprises one or more intraoral, extraoral or external sources from the group of electromagnetic radiation, magnetic resonance, positron-emitting radionuclide, and a single-photon emission tracer.

In another aspect, a detector for use in dental and medical x-ray imaging modalities comprises a converter and a collector, the detector configured to transform an x-ray beam into light and then convert the light into electrical signals at one or more photosensitive areas of the collector without the light first passing through the two or more wiring layers of the collector.

In a feature of this aspect, the detector further comprises one or more amplifiers sets of circuits that amplify the electrical signals within the detector before being processed by one or more analog-to-digital converters for single or parallel multiple sampling readout and one or more output streams for controlling the switching of sub streams at each frame via multiple exposure, dual PD, two-stage LOFICs, binary pixels, or combinations thereof, in the two or more wiring layers to allow a dual native gain while minimizing SNR and maintaining a WDR.

It is believed that one or more aspects and features described above provide a faster readout allowing for a reduction in the pulsing width during static and pulsed x-ray imaging modalities for radiation dose reduction as well as seamlessly switching from video to still picture and vice versa and slow-motion display. It also is believed that one or more aspects and features described above provide a dual native gain allowing for a reduction in mA to an even lower dose mode thereby increasing patient safety without compromising signal-to-noise and image quality.

It is further believed that the faster readout speed will allow a detector with higher frame rates, which helps for capturing images when a patient moves with no blurring, thereby avoiding the need to redo imaging if the patient didn't stay still. This helps a lot, especially with children. If doing fluoro, high speed procedures can be captured, like drilling on the tooth or bone without blurring. If doing CBCT, CT or Pano, the gantry can be rotated faster around the patient thereby reducing exposure time and radiation dosage. This also allows slow motion replay for teaching radiographic procedures to patients and students/residents. One or more aspects and features described herein are believed to provide processing light and electrical signals with an increased speed, even if the sensor includes higher megapixel counts, hence permitting reduction in the x-ray imaging detector's pixel size for early-stage disease diagnosis accuracy.

It also is believed that the dual native ISO/gain allows an additional gain mode for capturing images in lower light conditions, in this case an even lower radiation dose. Thus, the mA can be reduced even more than conventional and low-noise x-rays and videos-fluoro can still be achieved. This can be used for screening procedures such as lung cancer radiographies, CT, CBCT and mammography.

Additional features and embodiments that may utilize one or more of the aforementioned aspects of the invention are disclosed in the incorporated Uzbelger Feldman patents.

Still additional aspects, features, and embodiments of the invention are disclosed in provisional patent application 63/486,474, the disclosure of which is contained in the Appendix and incorporated herein by reference.

In addition to the aforementioned aspects and features of the present invention, it should be noted that the present invention further encompasses the various possible combinations and subcombinations of such aspects and features. Thus, for example, claims in this or a divisional or continuing patent application or applications may be separately directed to any aspect, feature, or embodiment disclosed herein, or combination thereof without requiring any other aspect, feature, or embodiment.

### BRIEF DESCRIPTION OF THE DRAWINGS

One or more preferred embodiments of the present invention now will be described in detail with reference to the accompanying drawings.

FIG. 1 is a schematic illustration of imaging apparatus and methods in accordance with a preferred embodiment of the invention.

FIG. 1A is a schematic illustration of steps of a method performed by a detector in imaging apparatus and methods in accordance with a preferred embodiment of the invention.

FIG. 1B is a schematic illustration of steps of a method performed by a computing device in imaging apparatus and methods in accordance with a preferred embodiment of the invention.

FIG. 2 is a schematic illustration of imaging apparatus and methods in accordance with a preferred embodiment of the invention.

FIG. 3 is a schematic illustration of a detector in imaging apparatus and methods in accordance with a preferred embodiment of the invention.

FIG. 4 is a schematic illustration of a front illuminated architecture including a wiring layer and photosensitive layer.

FIG. 5 is a schematic illustration of a back illuminated architecture including a wiring layer and photosensitive layer.

FIG. 6 is a schematic illustration of a back illuminated architecture representative of two or more wiring layers in accordance with preferred embodiments of the invention.

### DETAILED DESCRIPTION

As a preliminary matter, it will readily be understood by one having ordinary skill in the relevant art (“Ordinary Artisan”) that the present invention has broad utility and application. As should be understood, any embodiment may incorporate only one or a plurality of the above-disclosed aspects of the invention and may further incorporate only one or a plurality of the above-disclosed features. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features.

Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability in the United States of 35 U.S.C. § 112(f) with regard to claim construction, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each in general denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers,” “a picnic basket having crackers without cheese,” and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Additionally, as used herein “low dose” in the context of x-rays and gamma rays is intended to mean an x-ray or gamma ray beam comprising a low milliamperes setting below 2.5 to 15 mA for digital imaging standards of dental intraoral and extraoral radiography, 50 mA for mammography, 100 mA for stationary x-ray units, and 50 mA for CT scans.

Referring now to the drawings, one or more preferred embodiments of the present invention are next described. The following description of one or more preferred embodiments is merely exemplary in nature and is in no way intended to limit the invention, its implementations, or uses.

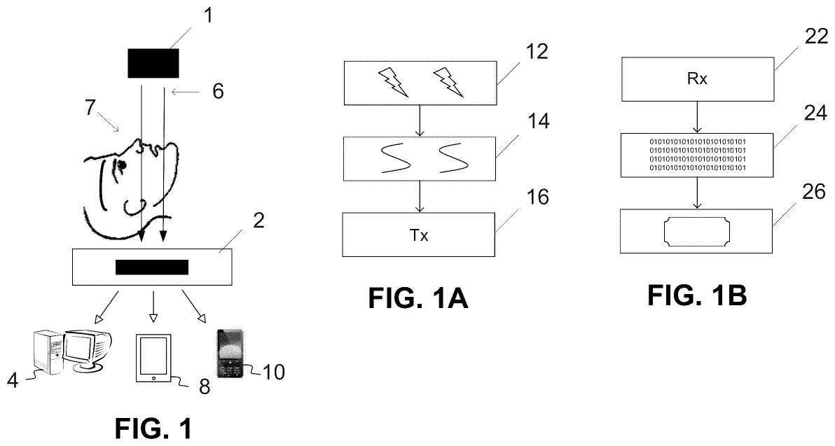

Turning now to FIG. 1, a schematic illustration of imaging apparatus and methods in accordance with a preferred embodiment of the invention are described. In this respect, a detector 2 may be used in all dental and medical x-ray imaging modalities, of which FIG. 1 is representative. As illustrated, an emitter 1 produces a beam 6 that travels through a patient examination area 7 and that is received at the detector 2.

The beam 6 emitted comprises gamma radiation or x-rays, both of which are referred to herein simply as x-rays. The emitter 1 preferably comprises one or more x-ray tubes, one or more gamma ray sources, or a combination thereof. With respect to the x-ray tubes, each x-ray tube preferably comprises a filament-based tube or a cold cathode-based tube such as a carbon nanotube having one or more focal spot sizes ranging from 0.001 microns to 3 mm. The nano focus and microfocus focal spot sizes will provide high resolution imaging at the cellular level for detecting early-stage disease. The detector 2 is shown in FIG. 1 as an extraoral or external detector. The detector 2 is used in x-ray imaging and may have a scintillator (indirect radiography) or may perform direct radiography or photon-counting with no scintillator.

The detector preferably comprises a stacked, back-illuminated sensor that comprises a charge coupled device (CCD), a complementary metal oxide semiconductor (CMOS), an active pixel sensor (APS) CMOS, an N-type metal-oxide-semiconductor (NMOS), a pinned photodiode (PPD), avalanche photodiodes (APDs), a single-photon avalanche diode (SPAD) imager, an APS thin film transistor (TFT) or single-crystalline silicon nanomembrane (Si NM), crystalline selenium (c-Se), or a combination thereof.

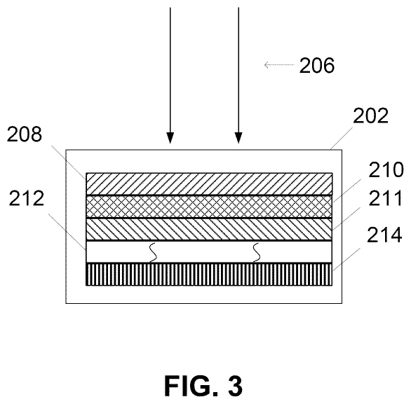

As schematically represented in FIG. 1A, certain preferred steps are performed within the detector 2, including a step 12 of transforming the beam 6 that is received at the detector 2 into light using a converter; a step 14 of capturing the light as it passes to a photosensitive layer of a collector within the detector, such as a photodiode layer, without first passing through any wiring layer, and to convert the light into electrical signals representative of digital images corresponding to the patient examination area 7, and further amplifying the electrical signals; and a step 16 of transmitting from the detector 2, based on the electrical signals, data representative of digital images for display of digital images to a user on a computing device. The detector 2 is further discussed in greater detail below with reference to FIG. 3.

The transforming of the beam into light may be performed by one or more organic x-ray converters, an organic photoconductive film (OPF), an organic photodetector (OPD), inorganic x-ray converters, or combination thereof, including Perovskite, halide Perovskite (inorganic, hybrid, organic-inorganic, 2/3D mixed dimensional and double Perovskite), lead halide Perovskite and single-crystalline Perovskite.

In a feature, the x-ray converter comprises a scintillator, a nanodots-based converter, or a combination thereof.

In a feature, the x-ray converter comprises a scintillator, a nanodots-based converter that is made from one of the groups of inorganic quantum dots, carbon-based quantum dots, perovskites quantum dots, and combinations thereof.

Computing devices of users are schematically shown in FIG. 1 as including a desktop or laptop computer 4, a tablet 8, and a smartphone 10. The transmission of the data that is performed at step 16 within the detector preferably is received by such a computing device. Specifically, FIG. 1B schematically shows certain steps preferably performed within such a computing device, including the step 22 of receiving at such computing device the data transmitted from the detector 2 at step 16; the step 24 of processing, noise filtering, reconstructing, and enhancing the received data; and the step 26 of displaying the 2D or 3D digital images to a user on a display of the computing device. The computing device to which the data is transmitted preferably is a desktop or laptop computer, a wireless mobile computing device, a tablet, or smartphone, VR glasses, a headset, or a hologram projector. Exemplary such computing devices are represented in FIG. 1.

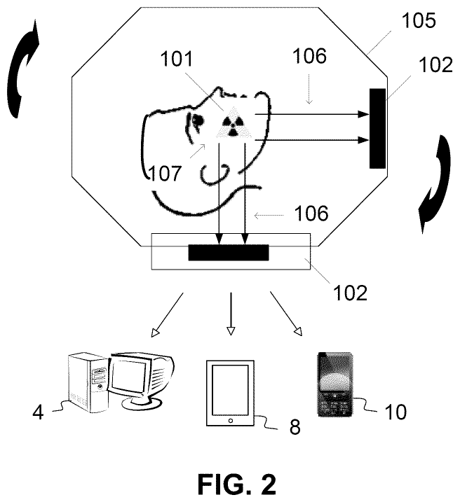

FIG. 2 is a schematic illustration of imaging apparatus and methods similar to the illustration of FIG. 1, but in which two or more detectors 102 mounted on a common support 105 for rotational movement around the patient examination area 107. The emitter 101 is shown in FIG. 2 as emitting radiation 106 and comprises an intraoral or internal source located inside of the patient that may include, for example, a miniature x-ray or gamma ray source, a positron-emitting radiotracer source, or a single-photon emission tracer.

It will be appreciated that while detector 2 and detectors 102 have been shown as extraoral or external detectors in the disclosed embodiments of FIGS. 1 and 2, intraoral or internal detectors can be used in other embodiments of the invention. Irrespective, in any such detector the light is collected at photosensitive areas without passing through any wiring layer. In accordance therewith, a beam 206 is converted into light and then focused, filtered, and collected at a photosensitive layer without passing through any wiring layers.

In particular, FIG. 3 is a schematic illustration of a detector used in imaging apparatus and methods in accordance with a preferred embodiment of the invention. The direction of travel of beam 206 comprising low-dose gamma rays or X-rays for detection by the detector is indicated by the arrows in FIG. 3. As shown in FIG. 3, the detector comprises a housing 202 in which is contained and enclosed one or more coating layers 208 including, for example, a grid, a refractive, a reflective, an anti-refractive or an antireflective layer; a converter 210, one or more focusing arrangements 211 including, for example, a radiation hardened or non-radiation hardened fiber optic plate or nano optic plate; a collector 212; and a transmitter 214. The one or more focusing arrangements 211 preferably comprises a microlens array, an anti-glare filter, a light intensity boost film, a light control film, a color filter, and combinations thereof.

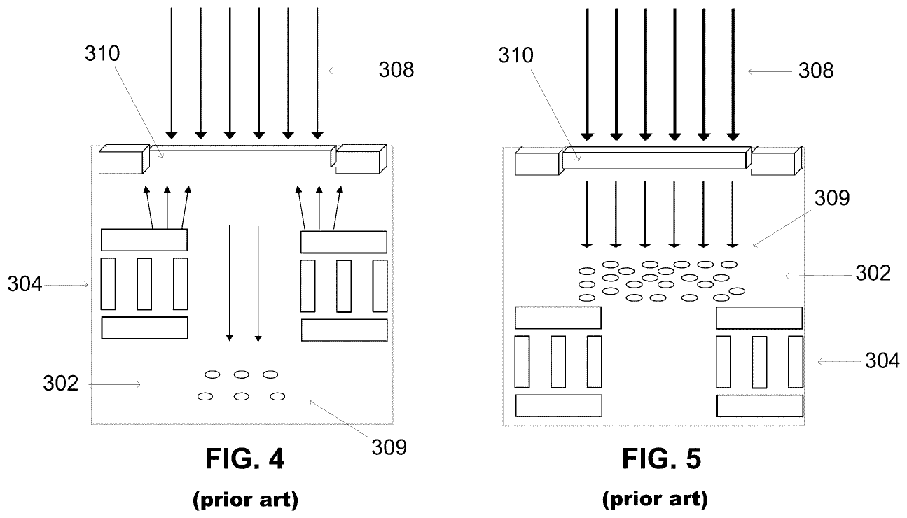

FIG. 4 schematically illustrates a front illuminated architecture of the prior art. This architecture of FIG. 4 includes a pixel gate 310, a wiring layer 304, and a photosensitive layer 302. The incident beam 308 encounters the pixel gate 310 and then the wiring layer 304 before encountering the photosensitive layer 302. Light is collected at area 309 behind wiring layer 304 relative to the direction of travel of light 308 in FIG. 4.

The collector 212 preferably has a pixel size from 0.001 microns to 500 microns.

FIG. 5 schematically illustrates a back illuminated architecture of the prior art. Like the architecture of FIG. 4, this architecture of FIG. 5 includes a pixel gate 310, a wiring layer 304, and a photosensitive layer 302. Unlike the architecture of FIG. 4, the incident beam 308 encounters the pixel gate 310 and then the photosensitive layer 302 without passing through any wiring layer 304. Thus, light is collected at area 309 in front of wiring layer 304 relative to the direction of travel of light 308 in FIG. 5.

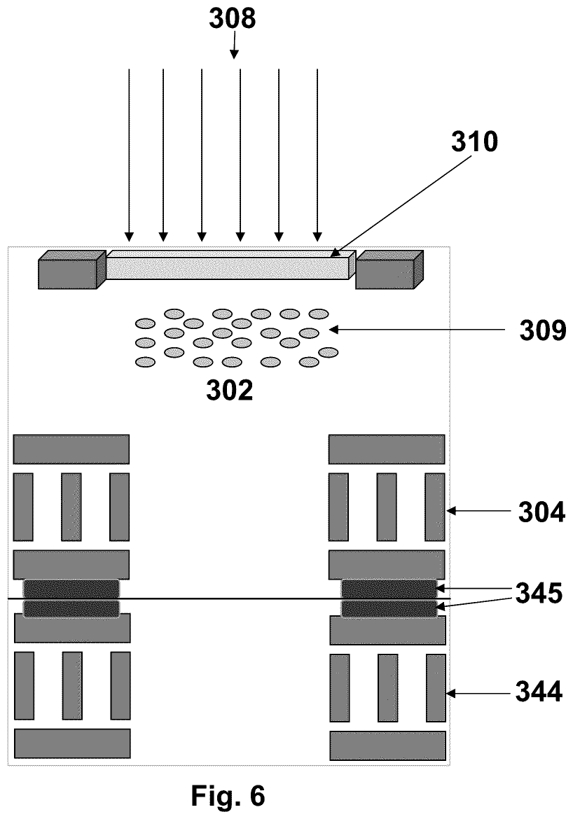

FIG. 6 schematically illustrates a stacked back illuminated architecture in accordance with one or more aspects and features of the present invention. Like the architecture of FIG. 5, the architecture of FIG. 6 includes a pixel gate 310, a wiring layer 304, and a photosensitive layer 302 at which light is collected in area 309. Furthermore, the incident beam 308 encounters the pixel gate 310 and then the photosensitive layer 302 without passing through any wiring layer 304. Thus, light is collected at area 309 in front of any wiring layer 304 relative to the direction of travel of light 308 in FIG. 6.

Unlike the architectures of FIG. 5 and FIG. 4, the architecture of FIG. 6 further includes a second wiring layer 344. The second wiring layer 344 is attached to the first wiring layer 304 through microbumps, direct bonding followed by Via-last through silicon via (Via-last TSV), hybrid bonding (HB) technologies, or a combination thereof at 345. Additional wiring layers may be attached in a similar manner in accordance with additional embodiments of the invention. As shown in FIG. 6, the plurality of wiring layers is oriented behind the photosensitive areas in the direction of travel of the light within the detector.

In further regard to this, a detector in accordance with the present invention comprises a plurality of wiring layers comprising stacked substrates attached together. Each substrate comprises one or more processing circuits. The additional wiring layers enable the detector to be configured for faster readout speeds and dual native ISO, which is believed to be a significant improvement over prior detectors in the context of dental and medical x-ray imaging modalities.

The stacked substrates of the wiring layers are attached together by microbumps, direct bonding followed by Via-last through silicon via (Via-last TSV) and hybrid bonding (HB) technologies. Each of the stacked substrates of the wiring layers comprise either a single wafer or a plurality of wafers that may be stitched, butted, or both. As shown in FIG. 6, the detector comprises two wiring layers, each comprising a said substrate with one or more processing circuits.

Preferably, the electrical signals into which the light is transformed are amplified by one or more sets of circuits before the signals are processed by one or more analog-to-digital converters for single or parallel multiple sampling readout and one or more output streams for controlling the switching of sub streams at each frame via multiple exposure, dual PD, two-stage LOFICs, binary pixels, or combinations thereof in the two or more wiring layers to allow a dual native gain while minimizing SNR and maintaining a WDR.

In preferred embodiments the plurality of wiring layers comprises the transmitter as well as one or more DRAM; WDR logic; readout circuitry; one or more analog-to-digital converters with single or hybrid column counter and a scalable low voltage signaling interface with an embedded clock (SLVS-EC) or a SLVS with a double data rate source-synchronous clock (DDR-SSC); a column parallel correlated multiple sampling (CMS) effects readout circuits with one or more output streams for controlling the switching of sub streams at each frame; one or more digital to analog converter (DAC); and, line buffers. For the stacked substrate of the SPAD, the wiring layers and pixel electronics could be a simple SPAD, a SPAD with a bit counter, a SPAD with a bit counter and a time-to-digital converter (TDC), or a combination thereof. The data is transmitted using the plurality of analog-to-digital converters for single or parallel multiple sampling readout and one or more output streams for controlling the switching of sub streams at each frame. The computing device to which the data is transmitted preferably is a desktop or laptop computer, a wireless mobile computing device, a tablet, or a smartphone. The computing device further may be VR glasses, a headset, or a hologram projector. The method also preferably comprises the steps of receiving the fast readout speed and dual native gain transmitted data and processing the data and displaying the digital images to a user on the computing device via a field-programmable gate array (FPGA) interface card(s) and protocol(s). The digital images displayed to a user may be 2D and 3D still images or real time video, and switching therebetween preferably is provided.

It is believed that one or more aspects and features described above provide a faster readout allowing for a reduction in the pulsing width during static and pulsed x-ray imaging modalities for radiation dose reduction as well as seamlessly switching from video to still picture and vice versa and slow-motion display. It also is believed that one or more aspects and features described above provide a dual native gain allowing for a reduction in mA to an even lower dose mode thereby increasing patient safety without compromising signal-to-noise and image quality.

It is further believed that the faster readout speed will allow a detector with higher frame rates, which helps for capturing images when a patient moves with no blurring, thereby avoiding the need to redo imaging if the patient didn't stay still. This helps a lot, especially with children. If doing fluoro, high speed procedures can be captured, like drilling on the tooth or bone without blurring. If doing CBCT, CT or Pano, the gantry can be rotated faster around the patient thereby reducing exposure time and radiation dosage. This also allows slow motion replay for teaching radiographic procedures to patients and students/residents. It is believed that one or more aspects and features described herein provide processing light and electrical signals with an increased speed, even if the sensor includes higher megapixel counts, resulting in a reduction of the x-ray imaging detector's pixel size for early-stage disease diagnosis accuracy.

It also is believed that the dual native ISO/gain allows an additional gain mode for capturing images in lower light conditions, in this case an even lower radiation dose. Thus, the mA can be reduced even more than conventional and low-noise x-rays and videos-fluoro can still be achieved. This can be used for screening procedures such as lung cancer radiographies, CT, CBCT, and mammography.

Preferred methods of the invention may be used in dental digital radiography; low-dose dental digital radiography; dental fluoroscopy; dental panoramic scanning; dental cephalometric scanning; dental cone beam computed tomography (CBCT); linear tomography; digital tomosynthesis; dental x-ray stereoscopic spectroscopy; photon-counting computed tomography; photon-counting radiography, positron emission tomography (PET); single-photon emission computed tomography (SPECT); and general radiography, fluoroscopy, mammography and computed tomography.

Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

For example, one or more of the emitters and detectors may be mounted to a wall or ceiling with appropriate supports or may be handheld and portable. Rotational support apparatus for the emitters and detectors also may be provided as disclosed, for example, in incorporated references, such as the Uzbelger Feldman '563 patent.

Additionally, it is contemplated that the stacked substrates of the wiring layers may comprise a radiation resistant chip. It also is contemplated that the x-ray converter may comprise a solid, liquid, gas, or combination thereof, a said x-ray converter is coupled to the collector; the x-ray converter is coupled to a plate and the plate is coupled to the collector; and combinations thereof.

It further is contemplated that the collector may act as an x-ray converter, such as in a direct radiography approach, photon counting, or a combination thereof.

It is also contemplated that the detector may comprise a lightproof housing within which components of the detector are enclosed. The lightproof housing preferably comprises a radiation shielded back side that minimizes backscattered radiation.

In some embodiments, the detector may comprise a scalable tile detector, and in other embodiments the detector may comprise a flat panel detector.

It also is contemplated that a housing of the detector may comprise a coating layer that filters or reflects the light within the detector, wherein the coating layer is located on top of a converter.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof. Indeed, while preferred embodiments have been described in detail in the context of dentistry and medicine, the present invention is not limited to use only in such context, and other contexts of use include veterinary medicine, astronomy, industrial x-ray inspection, non-destructive testing, and airport security.

1. An imaging method, comprising the steps of:
(a) causing a beam to travel from an emitter through an examination area for receipt at a detector; and
(b) within the detector,
(i) transforming the beam that is received into light,
(ii) transforming the light into electrical signals representative of digital images corresponding to the examination area, including using a collector within the detector to collect the light as it passes to photosensitive areas of the collector without first passing through any wiring layer of the collector, and
(iii) transmitting from the detector the data representative of digital images for display of the digital images to a user on a computing device;
(c) wherein the detector comprises a plurality of wiring layers comprising stacked substrates attached together, each substrate comprising one or more processing circuits by which the detector is configured for fast readout speed and dual native ISO.

2. The imaging method of claim 1, wherein the detector comprises a charge coupled device (CCD), a complementary metal oxide semiconductor (CMOS), an active pixel sensor (APS) CMOS, an N-type metal-oxide-semiconductor (NMOS), a pinned photodiode (PPD), avalanche photodiodes (APDs), a single-photon avalanche diode (SPAD) imager, an APS thin film transistor (TFT) or single-crystalline silicon nanomembrane (Si NM), crystalline selenium (c-Se), or combination thereof.

3. The imaging method of claim 1, wherein the step of transforming the beam into light is performed by an organic x-ray converter, an organic photoconductive film (OPF), an organic photodetector (OPD), an inorganic x-ray converter, or combination thereof, including Perovskite, halide Perovskite (inorganic, hybrid, organic-inorganic, 2/3D mixed dimensional and double Perovskite), lead halide Perovskite and single-crystalline Perovskite.

4-6. (canceled)

7. The imaging method of claim 1, wherein the collector has a pixel size from 0.001 microns to 500 microns.

8. The imaging method of claim 1, wherein the step of transforming the beam into light is performed by an x-ray converter comprising a scintillator, a nanodots-based converter, or a combination thereof.

9. The imaging method of claim 8, wherein the nanodots-based converter is made from one of the groups of inorganic quantum dots, carbon-based quantum dots, perovskites quantum dots, or a combination thereof.

10. (canceled)

11. The imaging method of claim 1, wherein the imaging method is used in direct or indirect radiography.

12. The imaging method of claim 1, wherein the stacked substrates of the wiring layers are attached together by microbumps, direct bonding followed by Via-last through silicon via (Via-last TSV) technologies, and hybrid bonding (HB) technologies.

13. The imaging method of claim 1, wherein each of the stacked substrates of the wiring layers comprise single wafers that are stitched, butted, or both.

14-15. (canceled)

16. The imaging method of claim 1, wherein the plurality of wiring layers is oriented behind one or more photosensitive areas in the direction of travel of the light within the detector.

17. The imaging method of claim 1, wherein the stacked substrates of the wiring layers comprise one or more DRAM; WDR logic; readout circuitry; one or more analog-to-digital converters with single or hybrid column counter and a scalable low voltage signaling interface with an embedded clock (SLVS-EC) or a SLVS with a double data rate source-synchronous clock (DDR-SSC); a column parallel correlated multiple sampling (CMS) effects readout circuits with one or more output streams for controlling the switching of sub streams at each frame; one or more digital to analog converter (DAC); and, line buffers.

18. The imaging method of claim 1, wherein the stacked substrates of the wiring layers comprise single-photon avalanche diode (SPAD) arrays.

19. The imaging method of claim 18, wherein the SPAD arrays comprise a bit counter.

20. The imaging method of claim 19, wherein the SPAD arrays comprise a time-to-digital converter (TDC) or a combination thereof.

21. The imaging method of claim 1, wherein the detector comprises one or more field-programmable gate array (FPGA) interface cards for transmitting from the detector the data representative of digital images for display of the digital images to a user on a computing device.

22. The imaging method of claim 1, wherein the stacked substrates of the wiring layers comprise a plurality of analog-to-digital converters, and wherein the data is transmitted using the plurality of analog-to-digital converters for single or parallel multiple sampling readout and one or more output streams for controlling the switching of sub streams at each frame.

23. The imaging method of claim 22, wherein the computing device to which the data is transmitted is a desktop or laptop computer; a wireless mobile computing device; a tablet; a smartphone; VR glasses; a headset; or a hologram projector.

24-25. (canceled)

26. The imaging method of claim 1, further comprising aiming the light to the photosensitive areas of the collector within the detector.

27. The imaging method of claim 16, wherein the aiming of the light is performed using a microlens array, an anti-glare filter, a light intensity boost film, a light control film or a color filter, a radiation hardened or non-radiation hardened fiber optic plate or nano optic plate, or combination thereof.

28-32. (canceled)

33. A low-dose radiation imaging apparatus, comprising:
(a) one or more emitters each having one or more focal spot sizes ranging from 0.001 microns to 3 mm and being configured to emit a low-dose gamma or x-ray beam through a patient examination area; and
(b) one or more detectors each configured to receive a said beam;
(c) wherein each detector comprises a housing containing:
(i) a collector that converts the light into electrical signals representative of digital images corresponding to the patient examination area, wherein the light that is collected passes to one or more photosensitive areas of the collector without passing through any wiring layer of the collector, and
(ii) a transmitter that transmits from the detector the data representative of digital images for display of the digital images to a user on a computing device via one or more field-programmable gate array (FPGA) interface cards and protocols,
(iii) wherein the two or more wiring layers comprise the transmitter and further comprise one or more DRAM, WDR logic, readout circuitry, one or more analog-to-digital converters with single or hybrid column counter with a scalable low voltage signaling interface with an embedded clock (SLVS-EC) or a SLVS with a double data rate source-synchronous clock (DDR-SSC), a column parallel correlated multiple sampling (CMS) effects readout circuit with one or more output streams for controlling switching of sub streams at each of a plurality of frames, digital to analog converter (DAC), and line buffers, whereby a large number of frames per second and greater image acquisition is achieved with a reduction in radiation dosage, a blur effect from moving objects and subjects during a radiographic examination or fluoroscopic procedures is minimized, seamlessly switching from video to still picture and vice versa, and slow-motion displaying are provided while allowing developing detectors with higher megapixel counts, and
(iv) wherein the detector comprises a plurality of wiring layers comprising stacked substrates attached together, each substrate comprising one or more processing circuits including amplifiers and one or more analog-to-digital converters, the amplifiers amplifying the electrical signals within the detector before being processed by the one or more analog-to-digital converters for single or parallel multiple sampling readout and one or more output streams for controlling the switching of sub streams at each frame via multiple exposure, dual PD, two-stage LOFICs, binary pixels, or combinations thereof, to allow a dual native gain while minimizing SNR and maintaining a WDR.

34-37. (canceled)
